(19) United States
(12) Patent Application Publication
(10) Pub. No.: US 2025/0275728 A1
(43) Pub. Date: Sep. 4, 2025

(54) MEDICAL IMAGING SYSTEM WITH A MAIN RAIL SYSTEM AND A SECURING RAIL SYSTEM

(71) Applicant: Siemens Healthineers AG, Forchheim (DE)

(72) Inventors: Stefan GROSS, Trabitz (DE); Rita KRUG, Fuerth (DE)

(73) Assignee: Siemens Healthineers AG, Forchheim (DE)

(21) Appl. No.: 19/063,807

(22) Filed: Feb. 26, 2025

(30) Foreign Application Priority Data
Feb. 29, 2024 (DE) 10 2024 201 889.9

(51) Int. Cl. A61B 6/03; A61B 6/00

(52) U.S. Cl. CPC A61B 6/035; A61B 6/4435

(57) ABSTRACT

A computed tomography gantry is movably mounted via a carriage and a main rail system such that a translational motion of the computed tomography gantry is executable along the main rail system. The carriage and the main rail system are configured to transfer a driving force for the translational motion of the computed tomography gantry non-positively from the carriage to the main rail system. The securing rail system includes at least one securing rail and at least one coupling element. The at least one securing rail is arranged on a floor section and is aligned with the main rail system. The at least one coupling element is arranged on the carriage or on the computed tomography gantry. The at least one coupling element is connected to the at least one securing rail via a rear grip and/or positively to secure against overturning of the computed tomography gantry.

### CROSS-REFERENCE TO RELATED APPLICATION(S)

The present application claims priority under 35 U.S.C. § 119 to German Patent Application No. 10 2024 201 889.9, filed Feb. 29, 2024, the entire contents of which is incorporated herein by reference.

### FIELD

One or more example embodiments of the present invention relate to a medical imaging system with a main rail system and a securing rail system. Further, one or more example embodiments of the present invention relate to a method for moving and/or securing a computed tomography gantry.

### BACKGROUND

In the framework of computed tomography (CT), the length position of the gantry is precisely changed during the scan of an examination object. This is based on a continuous translational motion which is continuously detected for the imaging system. The accuracy of this motion and the position detection is decisive for the image quality, with sub-millimeter accuracy often being required.

Conventional positive drives, such as rack and pinion drives or belt drives, are frequently used and require a structured area of intervention which is difficult to clean and creates trip hazards as well as mechanical sensitivities.

A computed tomography system (CT system) is sometimes used for two adjacent treatment rooms. The reasons for this are firstly, the cost aspect in order not to have to install a CT system for each of the two rooms. Secondly, a mobile CT system which can be moved into a different room also offers advantages in relation to the required space.

When two treatment rooms which face each other are being attended to, the patient couches can be arranged, for example, head-to-head. The CT system can thus travel back and forth between the two patient couches, so the computed tomography gantry is arranged in the one treatment room with one side pointing toward the patient couch and is arranged in the facing treatment room with the other side pointing toward the patient couch. These different arrangements of the computed tomography gantry relative to the respective patient couch should be taken into account when processing the imaging data.

In some CT systems the scanning plane is substantially centrally or centrically arranged in the housing of the computed tomography gantry. However, in some CT systems the scanning plane is not arranged centrally in the housing of the computed tomography gantry. There is therefore a front of the computed tomography gantry and a back of the computed tomography gantry. For attending to two treatment rooms which face each other it would then be advantageous to always orient the computed tomography gantry with the front pointing toward the respective patient couch. For a two-room solution with two patient couches arranged head-to-head it must therefore be possible for the computed tomography gantry to rotate about 180° in order to be oriented with its front pointing toward the respective patient couch in each case.

Movable CT systems, in particular rail-guided and/or rotatable CT systems, can easily overturn. In particular, in the event of earthquakes and/or start-up problems during the translational motion can cause such overturning. Further, the high center of gravity of the CT system promotes the susceptibility to overturning.

### SUMMARY

An object of one or more embodiments of the present invention is to provide an earthquake-proof medical imaging system and/or one which is secured against overturning, with a movable computed tomography gantry.

Each subject matter of an independent claim achieves at least this object. The dependent claims take into account further advantageous aspects of embodiments of the present invention.

Independent of the grammatical term usage, individuals with male, female or other gender identities are included within the term.

An embodiment of the present invention relates to a medical imaging system, having a computed tomography gantry, a carriage, a main rail system and a securing rail system,
wherein the computed tomography gantry is movably mounted via the carriage and the main rail system in such a way that a translational motion of the computed tomography gantry can be executed along the main rail system,
wherein the carriage and the main rail system are configured to transfer a driving force for the translational motion of the computed tomography gantry non-positively from the carriage to the main rail system,
wherein the securing rail system comprises at least one securing rail and at least one coupling element, wherein the at least one securing rail is arranged on a floor section and is aligned with the main rail system, wherein the coupling element is arranged on the carriage or on the computed tomography gantry, wherein the coupling element is connected via a rear grip and/or positively to the securing rail to secure against overturning of the computed tomography gantry.

In particular, it can be provided that the main rail system and/or the securing rail system is/are stationary relative to a base area and/or is permanently anchored relative to the base area. In particular, it can be provided that an examination object is stationary relative to the main rail system and/or the securing rail system and/or relative to the base area. The main rail system can form, in particular, a linear guide for the carriage.

For example, the medical imaging system can have an examination table for positioning the examination object. The examination table can be stationary, in particular relative to the main rail system and/or the securing rail system and/or relative to the base area, and/or be permanently anchored relative to the main rail system and/or the securing rail system and/or relative to the base area. The examination object can be, for example, a person to be examined, in particular a patient, and/or be positioned on the examination table, in particular be positioned in a stationary manner relative to the examination table.

The translational motion can take place, in particular, relative to the main rail system and/or the securing rail system, relative to the base area, relative to the examination table and/or relative to the examination object. The translational motion can be, in particular, substantially horizontal. The base area can be, in particular, substantially horizontal. The base area can be, in particular, a floor of an examination room.

The computed tomography gantry can have, for example, a supporting frame and a rotor mounted so it can rotate relative to the supporting frame, with the radiation source and the radiation detector being arranged on the rotor. The computed tomography gantry can optionally have a tilting frame mounted to it can tilt relative to the supporting frame, with the rotor being arranged on the tilting frame. The radiation source and the radiation detector can interact to record a projection dataset of the examination object. The computed tomography gantry can have, for example, an opening. In particular the rail system, the examination table and the opening can be arranged relative to one another in such a way that the examination table is introduced into the opening by the translational motion of the computed tomography gantry, in particular is introduced into the opening together with the examination object positioned on the examination table.

One embodiment provides that a set of wheel-rail rolling contacts is formed between the carriage and the main rail system, with the carriage and the main rail system being configured to non-positively transfer the driving force for the translational motion of the computed tomography gantry from the carriage to the rail system via the set of wheel-rail rolling contacts.

One embodiment provides that the set of wheel-rail rolling contacts bears the entire weight of the carriage and the computed tomography gantry, with each wheel-rail rolling contact, which is contained in the set of wheel-rail rolling contacts and bears at least some of the entire weight of the carriage and the computed tomography gantry, non-positively transferring, in particular by way of friction, at least some of the driving force for the translational motion of the computed tomography gantry.

In particular, it can be provided that at least some of the entire weight of the carriage and the computed tomography gantry is not insignificant, for example is greater than a tenth of the entire weight of the carriage and the computed tomography gantry. In particular, it can be provided that at least some of the driving force for the translational motion of the computed tomography gantry is not insignificant, for example is greater than a tenth of the entire driving force for the translational motion of the computed tomography gantry.

In particular, for the medical imaging system it is possible to rule out that there is a wheel-rail rolling contact which bears some of the entire weight of the carriage and the computed tomography gantry but does not transfer any of the driving force for the translational motion of the computed tomography gantry.

Compared to a friction wheel drive, the inventive solution basically makes it possible to transfer a greater driving force. The friction force available for the drive depends on the coefficient of friction and the normal force with which the friction wheel is loaded. In particular when the wheel is directly driven and the friction of the wheel-rail roll contacts is utilized, the entire weight of the carriage and the computed tomography gantry can be used as the normal force. With a friction wheel it is a matter of a weight distribution between the rail wheels and the friction wheel, so only some of the weight is available as the normal force.

One embodiment provides that the main rail system has a set of main rails, with the carriage having a set of wheels, with the set of wheels being arranged on the set of main rails so as to roll.

In particular, it can be provided that the set of main rails and the set of wheels form the set of wheel-rail rolling contacts. In particular, it can be provided that each main rail of the set of main rails is a round rail and/or that each wheel of the set of wheels is a concave roller and/or is embodied to roll on a round rail. The main rails and/or the wheels can be produced from steel, for example. In particular, the securing rail system comprises at least one securing rail. The securing rail is embodied, for example, as a round rail and/or produced from steel.

The round rails can, in particular, be integrated in the floor without covering and without drive elements and in the process allow patient beds and instrument tables to run over them. The driving force for the translational motion of the computed tomography gantry can be transferred from the carriage to the main rail system, for example on the basis of a non-positive connection, in particular friction locking, between the wheels of the set of wheels and the main rails of the set of main rails.

One embodiment provides that for each wheel of the set of wheels, the carriage has a wheel-direct drive which interacts with this wheel and contributes proportionally to the driving force for the translational motion of the computed tomography gantry.

In particular, it can be provided that for each wheel of the set of wheels, the wheel-direct drive, which interacts with this wheel, directly drives this wheel and consequently contributes proportionally to the driving force for the translational motion of the computed tomography gantry. In particular, it can be provided that the driving force for the translational motion of the computed tomography gantry is generated by the wheel-direct drives of the wheels of the set of wheels together. The wheel-direct drive can have, for example, an electric motor, in particular an electric wheel hub motor.

The securing rail system comprises at least one securing rail. The at least one securing rail can be embodied as a round rail, a flat guide rail and/or pure profile guiding rail. The securing rails are preferably formed from a metal, such as steel or aluminum. Alternatively, the securing rails can be formed from a composite material or an alloy.

The at least one, preferably all, securing rails are aligned, in particular parallel, to the main rail system and/or the main rails of the main rail system. The main rails extend, for example, in a longitudinal direction, with the longitudinal direction being aligned with translational motion. The securing rails extend, for example, in the longitudinal direction. In particular, the securing rails are arranged in the same horizontal plane as the main rails. The at least one securing rail is arranged on a floor section. Preferably, the at least one securing rail is anchored to the floor section, in particular the subfloor, and/or is mechanically connected to it.

The securing rail system comprises at least one coupling element. The coupling element is preferably arranged on the carriage and/or the computed tomography gantry, in particular is mechanically connected and/or anchored to it. The coupling element preferably has a vertical extension. The coupling element is connected at one vertical end to the carriage and/or the computed tomography gantry, for example, and/or is oriented at the other vertical end toward the securing rail. The coupling element is preferably rigid and/or is based on a metallic material.

The coupling element is captively and/or positively connected, in particular via a rear grip, to the at least one securing rail. For example, the coupling element is connected to the securing rail via the end oriented toward the securing rail. The captive, positive and/or rear-grip connection between coupling element and securing rail can be moved, in particular, in the longitudinal direction and/or by executing the translational motion and/or can be moved along the securing rail. In particular, positive, captive and/or rear-grip connection and/or the position of this connection follows the translational motion. The computed tomography gantry is secured against falling over by the captive, positive and/or rear-grip connection. In the case where the computed tomography gantry tries to overturn, and, for example, the connection to the main rails becomes detached, the computed tomography gantry is held by the rear-grip and/or positive connection of the coupling element and the securing rail.

One embodiment of the present invention provides that the securing rail system comprises exactly one securing rail, wherein the main rail system comprises a plurality of main rails. Preferably, the main rail system comprises an even number of main rails, in particular two or four main rails.

The main rails are aligned and/or parallel in the transverse direction which is perpendicular to the longitudinal extension of the main rails. The main rails of the main rail system are mirror-symmetric to a mirror symmetry axis arranged in the longitudinal direction. The embodiment provides that the exactly one securing rail forms the mirror symmetry axis. This embodiment is based on the consideration of allowing a computed tomography gantry which can be rotated about 180 degrees and/or carriage which can be rotated about 180 degrees, without having to alter the main rails and/or the securing rail.

A further embodiment of the present invention provides that the securing rail system comprises a plurality of securing rails, in particular an integral number of securing rails. The securing rails are aligned and/or parallel in the transverse direction which is perpendicular to the longitudinal extension of the main rails. The main rail system comprises a plurality of main rails. Preferably, the main rail system comprises an even number of main rails, in particular two or four main rails. The main rails are aligned and/or parallel in the transverse direction which is perpendicular to the longitudinal extension of the main rails. The securing rails and the main rails are mirror-symmetric in respect of a joint mirror axis, with the mirror axis being aligned with the securing rails and the main rails. This embodiment is based on the consideration of allowing a computed tomography gantry which can be rotated about 180 degrees and/or carriage which can be rotated about 180 degrees without having to alter the main rails and/or the securing rail.

It is particularly preferable for the securing rail to comprise a slot. The slot is embodied and/or arranged to receive the coupling element positively and/or with a rear-grip. The slot can form, for example, a guide for the coupling element. The slot forms, for example, an elongate recess and/or indentation for receiving the coupling element, in particular for captively receiving the coupling element. The coupling element can be embodied as a tongue and/or groove for the slot.

It is optionally provided that the medical imaging system comprises a pivot bearing and a lifting apparatus,
wherein in a translational operating state of the medical imaging system, the carriage is mounted so it can move along the main rail system in such a way that a first translational motion of the carriage can be executed along the main rail system, wherein the computed tomography gantry, the pivot bearing and the lifting apparatus are each received in the carriage in such a way that they follow the first translational motion of the carriage,
wherein in a transitional operating state of the medical imaging system, the carriage is mounted via the lifting apparatus so it can move along a vertical rotational axis relative to the main rail system in such a way that a lifting movement of the carriage can be executed along the vertical rotational axis relative to the main rail system, wherein the computed tomography gantry is received in the carriage in such a way that it follows the lifting movement of the carriage relative to the main rail system,
wherein in a rotational operating state of the medical imaging system, the carriage is lifted by the lifting apparatus along the vertical rotational axis relative to the main rail system in such a way that the carriage is released from the main rail system, and the carriage is mounted so it can rotate via the pivot bearing about the vertical rotational axis relative to the main rail system in such a way that a rotational movement of the carriage can be executed about the vertical rotational axis relative to the main rail system, wherein the computed tomography gantry is received in the carriage in such a way that it follows the rotational movement of the carriage about the vertical rotational axis relative to the main rail system.

In particular, it can be provided that in the transitional operating state of the system, the carriage is mounted via the lifting apparatus so it can move along the vertical rotational axis relative to the main rail system in such a way that a lowering movement of the carriage can be executed along the vertical rotational axis relative to the main rail system, wherein the computed tomography gantry is received in the carriage in such a way that it follows the lowering movement of the carriage relative to the main rail system.

In particular, it can be provided that the system has a transport drive and/or a rotary drive. The transport drive can be configured, in particular, to drive the first translational motion of the carriage and/or to drive the second translational motion of the carriage. The rotary drive can be configured, in particular, to drive the rotational movement of the carriage about the vertical rotational axis relative to the main rail system. The pivot bearing can be, for example, a rolling bearing, in particular an axial rolling bearing. Instead of providing a rotary drive the main rail system can also be configured to manually drive the rotational movement of the carriage about the vertical rotational axis relative to the main rail system, for example due to the physical strength of an operator.

The lifting apparatus can have, for example, a set of lifting cylinders and/or a lift drive. In particular, it can be provided that the pivot bearing is arranged between two lifting cylinders of the set of lifting cylinders in relation to a horizontal direction and/or that the lifting cylinders of the set of lifting cylinders are synchronized with one another, for example via a shaft. The horizontal direction can, in particular, be substantially perpendicular to the first translational motion.

For example, the computed tomography gantry can thereby be rotatably arranged in such a way, in particular rotatable about 180°, in such a way that in two treatment rooms which face one another, each of two patient couches arranged head-to-head can be approached with the front of the computed tomography gantry in each case. The two treatment rooms would then be equal in relation to the generation of the imaging data insofar as the orientation of the computed tomography gantry relative to the respective patient couch is concerned.

The first translational motion can, in particular, be horizontal and/or parallel to the base area. The second translational motion can, in particular, be horizontal and/or parallel to the base area. The first translational motion can take place, for example, on a straight first path and/or on a curved first path. The second translational motion can take place, for example, on a straight second path and/or on a curved second path. The rail system can be configured, in particular, for a translation of the carriage between two treatment rooms.

The system can have, in particular, a cable guide which is configured to connect the computed tomography gantry and/or the carriage to an energy transmission facility, which is stationary relative to the base area, and/or to a data transmission facility, which is stationary relative to the base area, in particular during the first horizontal translational motion of the carriage and/or during the rotational movement of the carriage relative to the rail system. The cable guide can be, for example, ceiling-based and/or floor-based. The cable guide can have, for example, a cable column permanently connected to the carriage and/or to the computed tomography gantry and which is flexibly connected to the energy transmission facility, which is stationary relative to the base area, and/or to the data transmission facility, which is stationary relative to the base area. The system can have, for example, a transmission interface for transmission, in particular for bidirectional transmission, of energy and/or data between the carriage and the computed tomography gantry. The transmission interface can be, for example, contact-based and/or contactless.

In particular, the positive connection and/or connection via a rear grip between coupling element and securing rail is present via a rear grip and/or positively in the translational operating state, in the transitional operating state and rotational operating state. In other words, the coupling element is permanently and/or releasably connected to the securing rail. This embodiment is based on the consideration of also providing the secondary connection to the securing rail during the rotation, the raising or lift and/or the translational motion, so the system is always secured against overturning.

In particular, the coupling element is arranged in the rotation center of the computed tomography gantry and/or in the pivot point of the pivot bearing. In particular, the coupling element is arranged such that when the gantry rotates, the coupling element remains stationary. Further, it can be provided that the coupling element is arranged, at least in sections, along the rotational axis. For example, the coupling element has an intermediate section, with the intermediate section being arranged between securing rail and carriage or computed tomography gantry. Preferably, the coupling element in the intermediate section is identical and/or parallel to the rotational axis.

Preferably, the coupling element comprises a connecting section and a positive-fit section. The positive-fit section has a lower end in the vertical direction, wherein the positive-fit section is held and/or connected positively and/or via a rear grip in the securing rail. The connecting section is arranged on the carriage and/or the computed tomography gantry, wherein the connecting section has a vertical extent and the positive-fit section is spaced apart from the carriage and/or the computed tomography gantry. Preferably, the intermediate section forms the vertical extension of the connecting section.

One embodiment of the present invention provides that the coupling element comprises an element pivot bearing. The element pivot bearing is embodied to allow, in particular guide and/or support, a rotation between positive-fit section and carriage and/or between positive-fit section and computed tomography gantry. The element pivot bearing is preferably embodied as a ball bearing or roller bearing. Alternatively, the element pivot bearing can be embodied as a sliding bearing, axial bearing or pivot bearing.

A further embodiment provides that the positive-fit section is shaped and/or embodied such that the positive-fit section connected and/or held positively and/or via a rear grip can rotate about a vertical axis. In particular, the positive-fit section can rotate in the securing rail. In particular, the positive-fit section is embodied and/or shaped such that the positive-fit section can rotate in the slot of the securing rail. For example, in a plan view the positive-fit section is round and/or circular, with the diameter of the round and/or circular positive-fit section being smaller than the diameter or the width of the slot of the securing rail.

In particular, it is provided that the positive-fit section is arranged positively, captively and/or with a rear grip in the slot of the securing rail, wherein the positive-fit section is shaped and/or embodied such that positive-fit section can rotate in the slot about a vertical axis.

A covering apparatus is optionally provided, wherein the covering apparatus is embodied to cover the securing rail upstream and/or downstream of the coupling element. In particular, the covering apparatus is embodied to cover the slot of the securing rail. In particular, covering apparatus has a cover strip for covering the securing rail. The cover strip can be embodied, for example, as a metal strip or plastics material strip. In particular, the cover strip can be based on a composite material. The cover strip is arranged, in particular, along the securing rail and/or longitudinal direction. The cover strip is preferably led through the coupling element, in particular captively. The coupling element is embodied, in particular, to raise the cover strip and/or expose the guide rail or slot for the positive-fit section.

A further subject matter of embodiments of the present invention is a method for moving a computed tomography gantry, wherein the computed tomography gantry is movably mounted via a carriage and a main rail system in such a way that a translational motion of the computed tomography gantry can be executed along the main rail system, wherein the computed tomography gantry is connected to a securing rail via a coupling element, wherein the at least one securing rail is arranged on a floor section and is aligned with the main rail system, wherein the method comprises:
executing the translational motion of the computed tomography gantry along the main rail system (L), wherein a driving force for the translational motion of the computed tomography gantry is non-positively transferred from the carriage to the main rail system (L),
securing the computed tomography gantry against overturning, wherein the coupling element is connected to the securing rail via a rear grip and/or positively.

Within the framework of the present invention, features, which are described in relation to different embodiments of the present invention and/or different categories of claim (method, use, apparatus, system, arrangement, etc.), can be combined to form further embodiments of the present invention. For example, a claim, which relates to a system, can also be developed with features, which are described or claimed in connection with a method, and vice versa. Functional features of a method can be executed by appropriately embodied, concrete components. The use of the indefinite article “a” or “an” does not preclude the relevant feature from also being present several times. Within the context of the present application the expression “based on” can, in particular, be understood within the meaning of the expression “using”. In particular, wording, according to which a first feature is calculated (alternative: ascertained, generated, etc.) on the basis of a second feature, does not preclude the first feature from also being calculated (alternative: ascertained, generated, etc.) on the basis of a third feature.

### BRIEF DESCRIPTION OF THE DRAWINGS

The present invention will be explained below on the basis of exemplary embodiments with reference to the accompanying Figures. The representation in the Figures is schematic, highly simplified and not necessarily to scale.

FIG. 1 shows a medical imaging system with a computed tomography gantry, a carriage and a main and a securing rail system in a translational operating state of the system.

FIG. 2 shows the medical imaging system with the computed tomography gantry, the carriage and the main and a securing rail system in a rotational operating state of the system.

FIG. 3 shows a medical imaging system without lifting-rotating module.

FIG. 4 shows the medical imaging system in a side view.

FIG. 5 shows a flowchart of a method for moving a computed tomography gantry.

### DETAILED DESCRIPTION

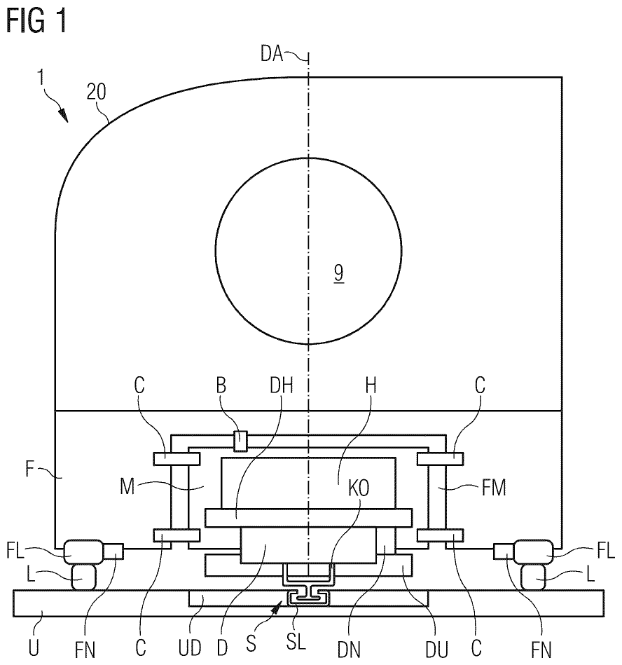

FIG. 1 shows the medical imaging system 1, having the computed tomography gantry 20, the carriage F, the main rail system L, the securing rail system S, the pivot bearing D and the lifting apparatus H in a translational operating state of the medical imaging system 1, with the carriage F being mounted in the translational operating state of the medical imaging system 1 so it can move along the main rail system L in such a way that a first translational motion of the carriage F can be executed along the main rail system L, with the computed tomography gantry 20, the pivot bearing D and the lifting apparatus H each being received in the carriage F in such a way that they follow the first translational motion of the carriage F. The computed tomography gantry 20 has the opening 9.

In a transitional operating state of the medical imaging system 1, the carriage F is mounted via the lifting apparatus H so it can move along a vertical rotational axis DA relative to the main rail system L in such a way that a lifting movement of the carriage F can be executed along the vertical rotational axis DA relative to the main rail system L, with the computed tomography gantry 20 being received in the carriage F in such a way that it follows the lifting movement of the carriage F relative to the main rail system L. The medical imaging system 1 has a transport drive FN and a rotary drive DN. The transport drive FN is configured for driving the first translational motion of the carriage F and/or for driving the second translational motion of the carriage F. The rotary drive DN is configured for driving the rotational movement of the carriage F about the vertical rotational axis DA relative to the main rail system L.

The securing rail system S comprises a securing rail SL and at least one coupling element KO. The securing rail is centrally arranged between the two rails of the main rail system L and extends parallel in the longitudinal direction to the rails of the main rail system L. The rail can be embodied, in particular, as a rail SL let into the floor, such as a slot-shaped recess between the two rails L, with the recess extending in the longitudinal direction. In particular, the securing rail SL is encompassed by a supporting structure DU, with the supporting structure being provided, in particular, when a lifting apparatus is used.

The coupling element KO is permanently or irreversibly connected to the carriage F, the gantry 20 or the lifting apparatus H and/or arranged on it. In particular, the coupling element KO is arranged on the lifting-rotating module M, the pivot bearing D or the base structure DU. The coupling element KO is centrally arranged between the two wheels FL and/or the rails L of the main rail system L. In particular, the coupling element is arranged with a vertical extension equal to the rotational axis DA and/or the coupling element KO is arranged, in a plan view from above, below the pivot bearing D. With a rotation of the computed tomography gantry 10, and therewith a changeover of the wheels FL, the coupling element remains substantially stationary and/or the coupling element remains connected to the securing rail SL.

To secure against overturning of the computed tomography gantry 20, the coupling element KO is connected via a rear grip and/or positively to the securing rail. For example, the securing rail comprises a slot, with the coupling element being arranged captively and/or with rear grip in the slot. For example, the rail SL and/or the slot has a collar for this purpose, which partially closes the slot and holds a section, arranged in the slot, of the coupling element KO in the slot. Despite captive and/or rear-grip connection to the securing rail, the coupling element KO can move in the longitudinal direction and/or along the longitudinal extension of the securing rail SL. In particular, the coupling element connected with a rear-grip and/or captively follows the translational motion. In particular, it is provided that the coupling element KO is still connected to the securing rail SL even during the lifting movement and/or a rotary movement of the computed tomography gantry 20. For this the lower end of the coupling element KO, which is arranged, for example, in the slot, can rotate about a vertical axis, in particular can rotate as a whole. Alternatively and/or in addition, the coupling element KO can comprise an element pivot bearing DE, so two sections of the coupling element can rotate relative to one another. This allows the coupling element KO to remain connected to the securing rail during a rotation of the computed tomography gantry 20.

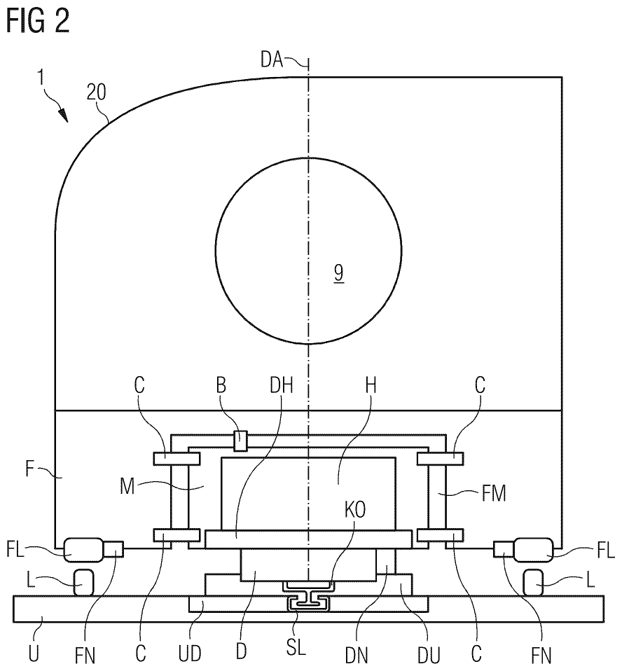

FIG. 2 shows the medical imaging system 1 having the computed tomography gantry 20, the carriage F, the main rail system L, the securing rail system S, the pivot bearing D and the lifting apparatus H in a rotational operating state of the medical imaging system 1, with the carriage F, in the rotational operating state of the medical imaging system 1, being raised via the lifting apparatus H along the vertical rotational axis DA relative to the rail system L in such a way that the carriage F is released from the main rail system L, and the carriage F being mounted via the pivot bearing D so it can rotate about the vertical rotational axis DA relative to the main rail system L in such a way that a rotational movement of the carriage F about the vertical rotational axis DA relative to the main rail system L can be executed, with the computed tomography gantry 20 being received in the carriage F in such a way that it follows the rotational movement of the carriage F about the vertical rotational axis DA relative to the main rail system L.

The main rail system L has a set of rails, with the carriage F having a set of wheels FL, with the rotational movement of the carriage F about the vertical rotational axis DA relative to the main rail system L taking place from a first angle about the vertical rotational axis DA to a second angle about the vertical rotational axis DA, with the set of wheels FL being arranged in relation to the vertical rotational axis DA in such a way that the set of wheels FL can roll on the set of rails if the carriage F is arranged relative to the main rail system L in the first angle about the vertical rotational axis DA, and that the set of wheels FL can roll on the set of rails if the carriage F is arranged relative to the main rail system L in the second angle about the vertical rotational axis DA.

The medical imaging system 1 optionally also has the supporting structure UD, with the main rail system L being stationary relative to the supporting structure UD, with the carriage F, in the translational operating state of the medical imaging system 1, being mounted so it can move along the main rail system L relative to the supporting structure UD in such a way that along the main rail system L, the first translational motion of the carriage F can be executed relative to the supporting structure UD, with the carriage F, in the transitional operating state of the system 1 and in the rotational operating state of the medical imaging system 1, being supported on the supporting structure UD via the lifting apparatus H and the pivot bearing D.

The medical imaging system 1 has the base structure DU, with the base structure DU being mounted via the lifting apparatus H so it can move along the vertical rotational axis DA relative to the carriage F, with the base structure DU being mounted via the pivot bearing D so it can rotate about the vertical rotational axis DA relative to the carriage F, with the lifting apparatus H being configured to enlarge a spacing between the base structure DU and the carriage F along the vertical rotational axis DA, starting from the translational operating state of the medical imaging system 1, and by pressing the base structure DU against the supporting structure UD, to raise the carriage F along the vertical rotational axis DA relative to the main rail system L in such a way that the carriage F is released from the rail system L, with the pressing of the base structure DU against the supporting structure UD fixing the base structure DU against a change in angle about the vertical rotational axis DA relative to the supporting structure UD in such a way that the carriage F is mounted via the pivot bearing D so it can rotate about the vertical rotational axis DA relative to the supporting structure UD, so the medical imaging system 1 passes into the rotational operating state of the medical imaging system 1.

The pressing of the base structure DU against the supporting structure UD frictionally fixes the base structure DU against the change in angle about the vertical rotational axis DA relative to the supporting structure UD in such a way that the carriage F is mounted via the pivot bearing D so it can rotate about the vertical rotational axis DA relative to the supporting structure UD.

The top of the supporting structure UD is substantially planar. The bottom of the base structure DU is substantially planar. The pressing of the base structure DU against the supporting structure UD causes friction locking between the top of the supporting structure UD and the bottom of the base structure DU, with the base structure DU being frictionally fixed by the friction locking against the change in angle about the vertical rotational axis DA relative to the supporting structure UD in such a way that the carriage F is mounted via the pivot bearing D so it can rotate about the vertical rotational axis DA relative to the supporting structure UD.

The lifting apparatus H is configured, starting from the rotational operating state of the medical imaging system 1, to reduce the spacing between the base structure DU and the carriage F along the vertical rotational axis DA and thereby lower the carriage F relative to the main rail system L along the vertical rotational axis DA through to placing of the carriage F on the main rail system L, so the medical imaging system 1 passes into the translational operating state of the medical imaging system 1.

The lifting apparatus H and the pivot bearing D are directly coupled to one another, so they form a lifting-rotating module M, with the carriage F having a recess FM for receiving the lifting-rotating module M, with the lifting-rotating module M being arranged in the recess in such a way that the lifting apparatus H can protrude along the vertical rotational axis DA downwards out of the recess FM in order to raise the carriage F relative to the rail system L along the vertical rotational axis DA.

The lifting-rotating module M is detachably connected to the carriage F via a mechanical interface C and an electrical interface B. The lifting-rotating module M can consequently be separated from the carriage F or be mounted therein with little effort. The lifting-rotating module M can be embodied, in particular, in the form of a piston.

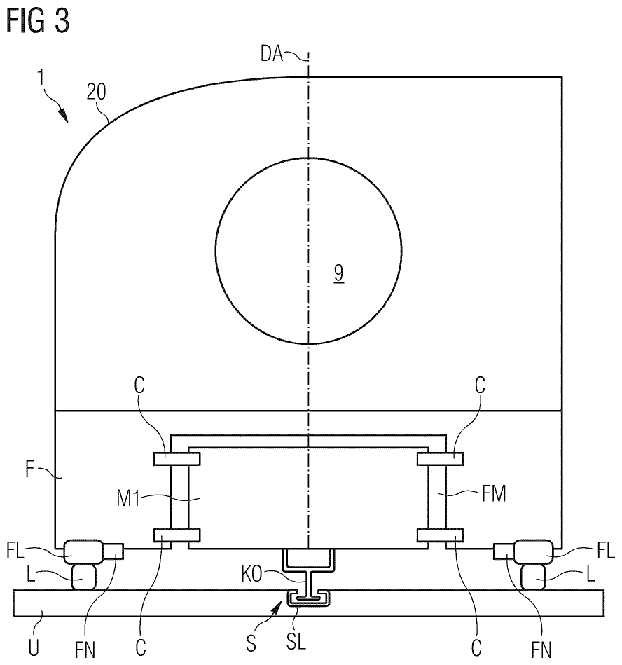

FIG. 3 shows a medical imaging system without the lifting-rotating module M. Instead of the lifting-rotating module M, only the structural element M1 is installed in the system. Instead of the structural element M1, this section of the carriage F can also be embodied to be continuous. The coupling element KO is arranged on the carriage For the structural element M1. The coupling element KO is embodied at the lower end in a T-shape or, in other words, it has a horizontal section and a vertical section there, with the horizontal section being arranged in the slot of the securing rail SL and being, for example, circular. The slot of the securing rail has a collar which narrows slightly upwards and/or partially closes the opening of the securing rail SL, so only the vertical section of the coupling element passes through and the horizontal section remains captive in the securing rail. The optional element pivot bearing DE can be arranged, for example, in the vertical section.

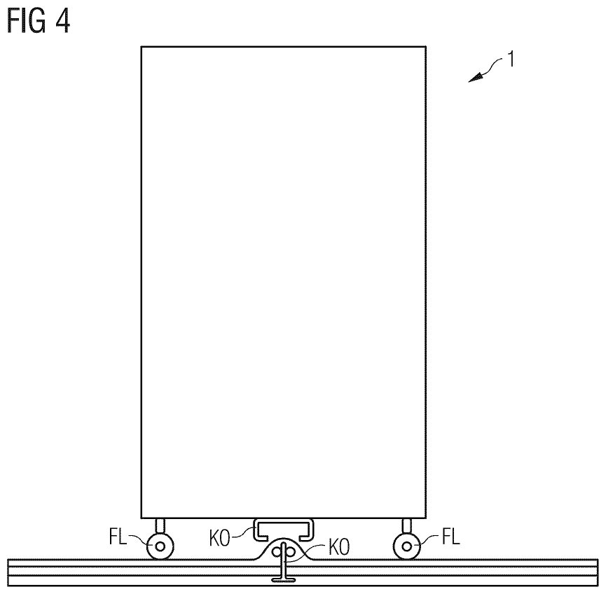

In order to illustrate an optional covering apparatus AV, FIG. 4 shows the medical imaging system 1 in a side view. The securing rail SL countersunk in the floor is covered in the longitudinal direction by a cover strip AB. The cover strip is preferably a metal strip. The coupling element KO has, preferably in an upper section, a lead-through for the cover strip AB, for example in the form of a recess. The cover strip is locally raised by the lead-through arranged above the securing rail SL and thus exposes the securing rail SL and/or its slot for the lower end of the coupling element KO. In the longitudinal direction upstream and downstream of the coupling element KO and/or the lead-through, the cover strip drops to floor level again.

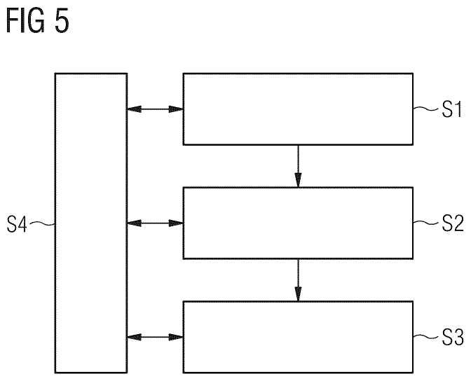

FIG. 5 shows a flowchart of a method for moving the computed tomography gantry 20, the method comprising:
executing S1 a first translational motion of a carriage F along a rail system L, while a system 1, which has the computed tomography gantry 20, the carriage F, the rail system L, a pivot bearing D and a lifting apparatus H, is in a translational operating state of the system 1, with the carriage F being mounted, in the translational operating state of the system 1, so it can move along the rail system L, with the computed tomography gantry 20, the pivot bearing D and the lifting apparatus H each being received in the carriage F in such a way that they follow the first translational motion of the carriage F,
executing S2 a lifting movement of the carriage F along a vertical rotational axis DA relative to the rail system L, while the system 1 is in a transitional operating state of the system 1, with the carriage F being mounted, in the transitional operating state of the system 1, via the lifting apparatus H so it can move along a vertical rotational axis DA relative to the rail system L, with the computed tomography gantry 20 being received in the carriage F in such a way that it follows the lifting movement of the carriage F relative to the rail system L,
executing S3 a rotational movement of the carriage F about the vertical rotational axis DA relative to the rail system L, while the system 1 is in a rotational operating state of the system 1, with the carriage F being raised, in the rotational operating state of the system 1, via the lifting apparatus H along the vertical rotational axis DA relative to the rail system L in such a way that the carriage F is released from the rail system L, and the carriage F is mounted via the pivot bearing D so it can rotate about the vertical rotational axis DA relative to the rail system L, with the computed tomography gantry 20 being received in the carriage F in such way that it follows the rotational movement of the carriage F about the vertical rotational axis DA relative to the rail system L.

The method comprises securing S4 the computed tomography gantry 20 against overturning. In this connection the coupling element KO is held and/or received captively and/or with rear grip by the securing rail SL. The securing S4 is permanent and/or simultaneous with the methods steps of executing S1 a first translational motion, executing S2 a lifting movement and executing S3 a rotational movement.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. A medical imaging system comprising:
a computed tomography gantry;
a carriage;
a main rail system; and
a securing rail system;
wherein the computed tomography gantry is movably mounted via the carriage and the main rail system such that the computed tomography gantry is configured to move translationally along the main rail system;
wherein the carriage and the main rail system are configured to transfer a driving force for translational motion of the computed tomography gantry non-positively from the carriage to the main rail system;
wherein the securing rail system includes at least one securing rail and at least one coupling element;
wherein the at least one securing rail is arranged on a floor section and is aligned with the main rail system;
wherein the at least one coupling element is arranged on the carriage or on the computed tomography gantry; and
wherein the at least one coupling element is connected to the at least one securing rail via at least one of a rear grip or positively to secure against overturning of the computed tomography gantry.

2. The medical imaging system as claimed in claim 1, wherein
the securing rail system includes exactly one securing rail,
the main rail system includes a plurality of main rails,
the securing rail forms a mirror symmetry axis of the plurality of main rails, and
the mirror symmetry axis is aligned with the securing rail.

3. The medical imaging system as claimed in claim 1, wherein
the securing rail system includes a plurality of securing rails,
the main rail system includes a plurality of main rails,
the plurality of securing rails and the plurality of main rails are mirror-symmetric with respect to a joint mirror axis, and
the joint mirror axis is aligned with the plurality of securing rails and the plurality of main rails.

4. The medical imaging system as claimed in claim 1, wherein
the at least one securing rail has a slot for at least one of positive or rear-grip receiving of the at least one coupling element, and
the at least one coupling element is configured to move translationally.

5. The medical imaging system as claimed in claim 1, further comprising:
a pivot bearing and a lifting apparatus, wherein
in a translational operating state of the medical imaging system,
the carriage is mounted to move along the main rail system such that a first translational motion of the carriage is executable along the main rail system, and
the computed tomography gantry, the pivot bearing and the lifting apparatus are each received in the carriage and follow the first translational motion of the carriage,
in a transitional operating state of the medical imaging system,
the carriage is mounted, via the lifting apparatus, to move along a vertical rotational axis relative to the main rail system such that a lifting movement of the carriage is executable along the vertical rotational axis relative to the main rail system, and
the computed tomography gantry is received in the carriage such that the computed tomography gantry follows the lifting movement of the carriage relative to the main rail system, and
in a rotational operating state of the medical imaging system,
the carriage is lifted via the lifting apparatus along the vertical rotational axis relative to the main rail system such that the carriage is released from the main rail system,
the carriage is mounted to rotate via the pivot bearing about the vertical rotational axis relative to the main rail system such that a rotational movement of the carriage is executable about the vertical rotational axis relative to the main rail system, and
the computed tomography gantry is received in the carriage such that the computed tomography gantry follows the rotational movement of the carriage about the vertical rotational axis relative to the main rail system.

6. The medical imaging system as claimed in claim 5, wherein at least one of the positive connection or the connection via the rear grip between the at least one coupling element and the at least one securing rail exists via at least one of a rear grip or positively in the translational operating state, the transitional operating state and the rotational operating state.

7. The medical imaging system as claimed in claim 5, wherein the at least one coupling element is arranged in a rotational center of the computed tomography gantry, at least one of in a pivot point of the pivot bearing or at least in sections along the vertical rotational axis.

8. The medical imaging system as claimed in claim 1, wherein
the at least one coupling element has a connecting section and a positive-fit section,
the positive-fit section includes a lower end in a vertical direction,
the positive-fit section is at least one of held or connected to the at least one securing rail at least one of positively or via the rear grip,
the connecting section is arranged on at least one of the carriage or the computed tomography gantry, and
the connecting section has a vertical extension and the positive-fit section is vertically spaced apart from the at least one of the carriage or the computed tomography gantry.

9. The medical imaging system as claimed in claim 8, wherein the at least one coupling element comprises:
an element pivot bearing, wherein
the element pivot bearing is configured to allow a rotation between the positive-fit section and the at least one of the carriage or the computed tomography gantry.

10. The medical imaging system as claimed in claim 8, wherein the positive-fit section is at least one of shaped or embodied such that the positive-fit section that is at least one of connected or held at least one of positively or via the rear grip is configured to rotate about a vertical axis.

11. The medical imaging system as claimed in claim 8, wherein
the positive-fit section is arranged at least one of positively, captively or with the rear grip in a slot of the at least one securing rail, and
the positive-fit section is at least one of shaped or embodied such that positive-fit section is configured to rotate in the slot about a vertical axis.

12. The medical imaging system as claimed in claim 1, further comprising:
a covering apparatus configured to cover the at least one securing rail at least one of upstream or downstream of the at least one coupling element.

13. The medical imaging system as claimed in claim 12, wherein the covering apparatus comprises:
a cover strip configured to cover the at least one securing rail, wherein
the cover strip is guided through the at least one coupling element.

14. A method for moving a computed tomography gantry, wherein the computed tomography gantry is movably mounted via a carriage and a main rail system such that a translational motion of the computed tomography gantry is executable along the main rail system, wherein the computed tomography gantry is connected to a securing rail via a coupling element, wherein the securing rail is arranged on a floor section and is aligned with the main rail system, and wherein the method comprises:
executing the translational motion of the computed tomography gantry along the main rail system, wherein a driving force for the translational motion of the computed tomography gantry is non-positively transferred from the carriage to the main rail system; and
securing the computed tomography gantry against overturning, wherein
the coupling element is connected to the securing rail via at least one of a rear grip or positively.

15. The medical imaging system as claimed in claim 2, further comprising:
a pivot bearing and a lifting apparatus, wherein
in a translational operating state of the medical imaging system,
the carriage is mounted to move along the main rail system such that a first translational motion of the carriage is executable along the main rail system, and
the computed tomography gantry, the pivot bearing and the lifting apparatus are each received in the carriage and follow the first translational motion of the carriage,
in a transitional operating state of the medical imaging system,
the carriage is mounted, via the lifting apparatus, to move along a vertical rotational axis relative to the main rail system such that a lifting movement of the carriage is executable along the vertical rotational axis relative to the main rail system, and
the computed tomography gantry is received in the carriage such that the computed tomography gantry follows the lifting movement of the carriage relative to the main rail system, and
in a rotational operating state of the medical imaging system,
the carriage is lifted via the lifting apparatus along the vertical rotational axis relative to the main rail system such that the carriage is released from the main rail system,
the carriage is mounted to rotate via the pivot bearing about the vertical rotational axis relative to the main rail system such that a rotational movement of the carriage is executable about the vertical rotational axis relative to the main rail system, and
the computed tomography gantry is received in the carriage such that the computed tomography gantry follows the rotational movement of the carriage about the vertical rotational axis relative to the main rail system.

16. The medical imaging system as claimed in claim 3, further comprising:
a pivot bearing and a lifting apparatus, wherein
in a translational operating state of the medical imaging system,
the carriage is mounted to move along the main rail system such that a first translational motion of the carriage is executable along the main rail system, and
the computed tomography gantry, the pivot bearing and the lifting apparatus are each received in the carriage and follow the first translational motion of the carriage,
in a transitional operating state of the medical imaging system,
the carriage is mounted, via the lifting apparatus, to move along a vertical rotational axis relative to the main rail system such that a lifting movement of the carriage is executable along the vertical rotational axis relative to the main rail system, and
the computed tomography gantry is received in the carriage such that the computed tomography gantry follows the lifting movement of the carriage relative to the main rail system, and
in a rotational operating state of the medical imaging system,
the carriage is lifted via the lifting apparatus along the vertical rotational axis relative to the main rail system such that the carriage is released from the main rail system,
the carriage is mounted to rotate via the pivot bearing about the vertical rotational axis relative to the main rail system such that a rotational movement of the carriage is executable about the vertical rotational axis relative to the main rail system, and
the computed tomography gantry is received in the carriage such that the computed tomography gantry follows the rotational movement of the carriage about the vertical rotational axis relative to the main rail system.

17. The medical imaging system as claimed in claim 6, wherein the at least one coupling element is arranged in a rotational center of the computed tomography gantry, at least one of in a pivot point of the pivot bearing or at least in sections along the vertical rotational axis.

18. The medical imaging system as claimed in claim 5, wherein
the at least one coupling element has a connecting section and a positive-fit section,
the positive-fit section includes a lower end in a vertical direction,
the positive-fit section is at least one of held or connected to the at least one securing rail at least one of positively or via the rear grip,
the connecting section is arranged on at least one of the carriage or the computed tomography gantry, and
the connecting section has a vertical extension and the positive-fit section is vertically spaced apart from the at least one of the carriage or the computed tomography gantry.

19. The medical imaging system as claimed in claim 9, wherein the positive-fit section is at least one of shaped or embodied such that the positive-fit section that is at least one of connected or held at least one of positively or via the rear grip is configured to rotate about a vertical axis.

20. The medical imaging system as claimed in claim 19, wherein
the positive-fit section is arranged at least one of positively, captively or with the rear grip in a slot of the at least one securing rail, and
the positive-fit section is at least one of shaped or embodied such that positive-fit section is configured to rotate in the slot about a vertical axis.
